(19) United States
(12) Patent Application Publication
(10) Pub. No.: US 2020/0014066 A1
(43) Pub. Date: Jan. 9, 2020

(54) NONAQUEOUS ELECTROLYTE SECONDARY BATTERY

(71) Applicant: Panasonic Intellectual Property Management Co., Ltd., Osaka-shi, Osaka (JP)

(72) Inventors: Masahiro Shiraga, Osaka (JP); Mitsuru Iwai, Osaka (JP); Yuanlong Zhong, Osaka (JP)

(73) Assignee: Panasonic Intellectual Property Management Co., Ltd., Osaka-shi, Osaka (JP)

(21) Appl. No.: 16/495,587

(22) PCT Filed: Jan. 26, 2018
(86) PCT No.: PCT/JP2018/002392

§ 371 Date: Sep. 19, 2019

(30) Foreign Application Priority Data
Mar. 24, 2017 (JP) 2017-059471

(51) Int. Cl. H01M 10/0567; H01M 10/0569; H01M 10/0568; H01M 10/0525; C07D 319/12; C07F 1/02

(52) U.S. Cl. CPC H01M 10/0567; H01M 10/0569; H01M 10/0568; H01M 2300/0034; C07D 319/12; C07F 1/02; H01M 10/0525

(57) ABSTRACT

This non-aqueous electrolyte secondary battery is provided with a positive electrode, a negative electrode and a non-aqueous electrolyte. The non-aqueous electrolyte contains: a non-aqueous solvent that contains a fluorine-containing cyclic carbonate; a cyclic carboxylic acid anhydride such as diglycolic acid anhydride; and an imide lithium salt having a sulfonyl group such as lithium bis(fluorosulfonyl)imide.

### TECHNICAL FIELD

The present invention relates to a technique concerning a non-aqueous electrolyte secondary battery.

### BACKGROUND ART

In recent years, a non-aqueous electrolyte secondary battery which includes a positive electrode, a negative electrode, and a non-aqueous electrolyte, and achieves charge and discharge by moving lithium ions between the positive and negative electrodes has widely been used as a secondary battery having a high output and a high energy density.

For example, Patent Literature 1 discloses a non-aqueous electrolyte secondary battery including: a positive electrode; a negative electrode; and a non-aqueous electrolyte containing a fluorine-containing cyclic carbonate. Patent Literature 1 describes that charge/discharge cycle characteristics of a non-aqueous electrolyte secondary battery at room temperature is improved by using the non-aqueous electrolyte containing a fluorine-containing cyclic carbonate.

### CITATION LIST

### Patent Literature

PATENT LITERATURE 1: Japanese Unexamined Patent Application Publication No. 2013-182807

### SUMMARY

However, the non-aqueous electrolyte secondary battery using a non-aqueous electrolyte containing a fluorine-containing cyclic carbonate has a problem that the capacity recovery rate after high-temperature storage decreases. The capacity recovery rate after high-temperature storage herein refers to the ratio of the battery capacity (recovered capacity) of a non-aqueous electrolyte secondary battery obtained when charge/discharge is performed again at room temperature (e.g. 25° C.) after storing the non-aqueous electrolyte secondary battery in a charged at a high temperature (e.g. 45° C. or more) for predetermined days to the battery capacity (capacity before storage) of the non-aqueous electrolyte secondary battery when charge/discharge is performed at room temperature (e.g. 25° C.), and is expressed by the following formula.

Capacity recovery rate after high-temperature storage=recovered capacity/capacity before storage×100

It is an advantage of the present disclosure to provide a non-aqueous electrolyte secondary battery that may suppress the decrease in the capacity recovery rate after high-temperature storage.

A non-aqueous electrolyte secondary battery of one aspect of the present disclosure comprises: a positive electrode, a negative electrode; and a non-aqueous electrolyte, wherein the non-aqueous electrolyte contains: a non-aqueous solvent containing a fluorine-containing cyclic carbonate; a cyclic carboxylic anhydride represented by the following formula (1); and an imide lithium salt having sulfonyl groups and represented by the following formula (2).

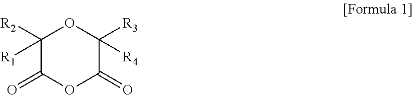

wherein R₁ to R₄ each independently represent H, an alkyl group, an alkene group, or an aryl group.

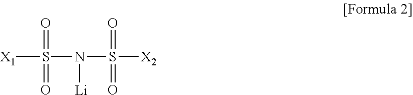

wherein X₁ to X₂ each independently represent a fluorine group or a fluoroalkyl group.

According to the non-aqueous electrolyte secondary battery of one aspect of the present disclosure, the decrease in the capacity recovery rate after high-temperature storage may be suppressed.

### DESCRIPTION OF EMBODIMENTS

In a conventional non-aqueous electrolyte secondary battery using a non-aqueous electrolyte containing a fluorine-containing cyclic carbonate, part of the fluorine-containing cyclic carbonate is decomposed on the negative electrode, for example, during charging/discharging and a film (SEI film) derived from the fluorine-containing cyclic carbonate is formed on the negative electrode. This film derived from the fluorine-containing cyclic carbonate has a function of suppressing further decomposition of the non-aqueous electrolyte on the negative electrode, but lacks thermal stability, and therefore the film is likely to be broken at a high-temperature environment. Accordingly, when the conventional non-aqueous electrolyte secondary battery using a non-aqueous electrolyte containing a fluorine-containing cyclic carbonate is stored at a high-temperature (e.g. 45° C. or more), the film derived from the fluorine-containing cyclic carbonate is broken and the decomposition of the non-aqueous electrode may progress during charging/discharging after the storage. As a result, the capacity of the non-aqueous electrolyte secondary battery after high-temperature storage decreases, so that the above-described decrease in the capacity recovery rate after high-temperature storage may be brought about. As a result of earnest studies, the present inventors have found that the decrease in the capacity recovery rate after high-temperature storage is suppressed by adding a cyclic carboxylic anhydride represented by the following formula (1) and an imide lithium salt having sulfonyl groups and represented by the following formula (2) to the non-aqueous electrolyte containing a fluorine-containing cyclic carbonate.

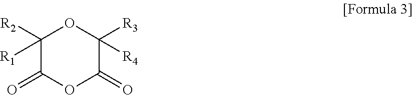

wherein R₁ to R₄ each independently represent H, an alkyl group, an alkene group, or an aryl group. The alkyl group is, for example, an alkyl group having 1 to 5 carbon atoms, such as a methyl group or an ethyl group, the alkene group is, for example, an alkene group having 2 to 5 carbon atoms, such as an ethylene group or a propylene group, and the aryl group is, for example, an aryl group having 6 to 10 carbon atoms, such as a phenyl group or benzyl group.

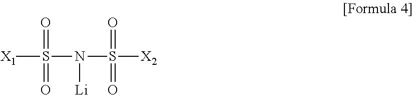

wherein X₁ to X₂ each independently represent a fluorine group or a fluoroalkyl group. The fluoroalkyl group is, for example, a fluoroalkyl group having 1 to 3 carbon atoms, such as a trifluoromethyl group or a pentafluoroethyl group.

This mechanism is not sufficiently clear but is inferred as follows. It is conceivable that in a non-aqueous electrolyte secondary battery using a non-aqueous electrolyte containing a fluorine-containing cyclic carbonate, the above imide lithium salt having sulfonyl groups, and the above cyclic carboxylic anhydride, a composite film is formed on the negative electrode during charging/discharging by decomposition of the above three substances. Since the composite film contains decomposed products of the imide lithium salt having sulfonyl groups and the cyclic carboxylic anhydride in addition to a decomposed product of the fluorine-containing carbonate, it is conceivable that the composite film is a film having a high thermal stability. As a result, even if the non-aqueous electrolyte secondary battery is stored at a high temperature, it can be considered that the breakage of the composite film is suppressed, and therefore the decomposition of the non-aqueous electrolyte is suppressed during charging/discharging after the storage. In addition, it can be considered that the composite film is a film having a high ion conductivity, and therefore if the composite film is formed on the negative electrode, an increase in the resistance value of the negative electrode is suppressed. From these, it is inferred that the decrease in the capacity recovery rate of the non-aqueous electrolyte secondary battery after high-temperature storage is suppressed. In addition, according to a non-aqueous electrolyte secondary battery of one aspect of the present disclosure, since the decomposition of the non-aqueous electrolyte due to high-temperature storage is suppressed, the amount of a gas to be produced accompanying the decomposition of the non-aqueous electrolyte can also be suppressed.

Hereinafter, an embodiment of the non-aqueous electrolyte secondary battery of one aspect of the present disclosure will be described. The embodiment described below is one exemplary embodiment, and the present disclosure is not limited to the embodiment.

The non-aqueous electrolyte secondary battery as one exemplary embodiment includes a positive electrode, a negative electrode, a separator, a non-aqueous electrolyte, and a battery case. Specifically, the non-aqueous electrolyte secondary battery has a structure in which a wound type electrode assembly having a positive electrode and a negative electrode each wound through a separator; and a non-aqueous electrolyte are housed in a battery case. The electrode assembly is not limited to the wound type electrode assembly, and other forms of electrode assemblies such as an electrode assembly obtained by laminating a positive electrode and a negative electrode with a separator interposed therebetween can be applied. The form of the non-aqueous electrolyte secondary battery is not particularly limited, and examples thereof include a cylindrical type, a rectangular type, a coin type, a button type, and a lamination type.

Hereinafter, the non-aqueous electrolyte, the positive electrode, the negative electrode, and the separator used for the non-aqueous electrolyte secondary battery as one exemplary embodiment will be described in detail.

[Non-Aqueous Electrolyte]

The non-aqueous electrolyte contains: a non-aqueous solvent containing a fluorine-containing cyclic carbonate: a cyclic carboxylic anhydride; and an imide lithium salt having sulfonyl groups. The non-aqueous electrolyte is not limited to a liquid electrolyte (non-aqueous electrolytic solution) but may be a solid electrolyte using a gel polymer or the like.

The fluorine-containing cyclic carbonate contained in the non-aqueous solvent is not particularly limited as long as it is a cyclic carbonate containing at least one atom of fluorine, and examples thereof include monofluoroethylene carbonate (FEC), 1,2-difluoroethylene carbonate, 1,2,3-trifluoropropylene carbonate, 2,3-difluoro-2,3-butylene carbonate, and 1,1,1,4,4,4-hexafluoro-2,3-butylene carbonate. These may be used singly or in combinations of two or more thereof. Among these, monofluoroethylene carbonate (FEC) is preferable from the viewpoint of suppressing the amount of hydrofluoric acid produced and other viewpoints.

For example, the content of the fluorine-containing cyclic carbonate in the non-aqueous solvent is preferably 5 vol % or more and 50 vol % or less, and more preferably 10 vol % or more and 20 vol % or less. If the content of the fluorine-containing cyclic carbonate in the non-aqueous solvent is less than 5 vol %, for example, the amount of a film to be produced, the film derived from the fluorine-containing cyclic carbonate, is small, compared to a case where the content of the fluorine-containing cyclic carbonate meets the above range, so that the charge/discharge cycle characteristics of the non-aqueous electrolyte secondary battery at room temperature may be deteriorated. If the content of the fluorine-containing cyclic carbonate in the non-aqueous solvent exceeds 50 vol %, for example, the thermal stability of the above composite film to be formed on the negative electrode is deteriorated, compared to a case where the content of the fluorine-containing cyclic carbonate meets the above range, so that the capacity recovery rate of the non-aqueous electrolyte secondary battery after high-temperature storage may be decreased.

The non-aqueous solvent may contain, for example, a non-fluorine solvent in addition to the fluorine-containing cyclic carbonate. Examples of the non-fluorine solvent include cyclic carbonates, chain carbonates, carboxylate esters, cyclic ethers, chain ethers, nitriles such as acetonitrile, amides such as dimethylformamide, and mixed solvents thereof.

Examples of the cyclic carbonates include ethylene carbonate (EC), propylene carbonate (PC), and butylene carbonate. Examples of the chain carbonates include dimethyl carbonate, ethyl methyl carbonate (EMC), diethyl carbonate, methyl propyl carbonate, ethyl propyl carbonate, and methyl isopropyl carbonate. These may be used singly or in combinations of two or more thereof.

Examples of the carboxylate esters include methyl acetate, ethyl acetate, propyl acetate, methyl propionate (MP), ethyl propionate, and γ-butyrolactone. These may be used singly or in combinations of two or more thereof.

Examples of the cyclic ethers include 1,3-dioxolane, 4-methyl-1,3-dioxolane, tetrahydrofuran, 2-methyltetrahydrofuran, propylene oxide, 1,2-butylene oxide, 1,3-dioxane, 1,4-dioxane, 1,3,5-trioxane, furan, 2-methylfuran, 1,8-cineole, and crown ethers. These may be used singly or in combinations of two or more thereof.

Examples of the chain ethers include 1,2-dimethoxyethane, diethyl ether, dipropyl ether, diisopropyl ether, dibutyl ether, dihexyl ether, ethyl vinyl ether, butyl vinyl ether, methyl phenyl ether, ethyl phenyl ether, butyl phenyl ether, pentyl phenyl ether, methoxytoluene, benzyl ethyl ether, diphenyl ether, dibenzyl ether, o-dimethoxybenzene, 1,2-diethoxyethane, 1,2-dibutoxyethane, diethylene glycol dimethyl ether, diethylene glycol diethyl ether, diethylene glycol dibutyl ether, 1,1-dimethoxymethane, 1,1-diethoxyethane, triethylene glycol dimethyl ether, and tetraethylene glycol dimethyl ether. These may be used singly or in combinations of two or more thereof.

The cyclic carboxylic anhydride contained in the non-aqueous electrolyte is not particularly limited as long as it is a substance represented by the above formula (1), and specific examples thereof include diglycolic anhydride, methyldiglycolic anhydride, dimethyldiglycolic anhydride, ethyldiglycolic anhydride, vinyldiglycolic anhydride, allyldiglycolic anhydride, and divinyldiglycolic anhydride. These may be used singly or in combinations of two or more thereof. Among these, diglycolic anhydride is preferable from the viewpoints such as enabling further suppression of the decrease in the capacity recovery rate of the non-aqueous electrolyte secondary battery after high-temperature storage.

The imide lithium salt having sulfonyl groups and contained in the non-aqueous electrolyte is not particularly limited as long as it is a substance represented by the above formula (2), and specific examples thereof include lithium bis(fluorosulfonyl)imide (LiFSI), lithium bis(trifluoromethanesulfonyl)imide (LiTFSI), lithium bis(pentafluoroethanesulfonyl)imide, and lithium bis (nonaflnorobutanesulfonyl)imide. These may be used singly or in combinations of two or more thereof. Among these, lithium bis(fluorosulfonyl)imide is preferable from the viewpoints such as enabling further suppression of the decrease in the capacity recovery rate of the non-aqueous electrolyte secondary battery after high-temperature storage.

The content of the cyclic carboxylic anhydride in the non-aqueous electrolyte and the content of the imide lithium salt having sulfonyl groups in the non-aqueous electrolyte are preferably in the following ranges from the viewpoint of enabling further suppression of the decrease in the capacity recovery rate of the non-aqueous electrolyte secondary battery after high-temperature storage, or from the viewpoints such as enabling further suppression of the gas production accompanying the high-temperature storage of the non-aqueous electrolyte secondary battery. The content of the cyclic carboxylic anhydride in the non-aqueous electrolyte is preferably 0.1 mass % or more and 1.5 mass % or less, and more preferably 0.2 mass % or more and 1 mass % or less. The content of the imide lithium salt having sulfonyl groups in the non-aqueous electrolyte is preferably 0.1 mass % or more and 1.5 mass % or less, and more preferably 0.2 mass % or more and 1 mass % or less.

The non-aqueous electrolyte may contain another lithium salt in addition to the imide lithium salt having sulfonyl groups. The lithium salt is a supporting electrolyte or the like generally used in a conventional non-aqueous electrolyte secondary battery, and examples thereof include LiPF₆, LiBF₄, LiAsF₆, LiClO₄, LiCF₃SO₃, Li[B(C₂O₄)₂], Li[B(C₂O₄)F₂], Li[P(C₂O₄)F₄], and Li[P(C₂O₄)₂F₂]. These lithium salts may be used singly or in combinations of two or more thereof.

[Positive Electrode]

The positive electrode includes, for example, a positive electrode current collector such as metal foil and a positive electrode active material layer formed on the positive electrode current collector. Foil of a metal, such as aluminum, that is stable in the electric potential range of the positive electrode, a film with such a metal disposed as an outer layer, and the like can be used for the positive electrode current collector. The positive electrode active material layer contains, for example, a positive electrode active material, a binder, an electrical conductor, and the like.

The positive electrode is obtained by, for example, applying/drying a positive electrode mixture slurry containing the positive electrode active material, the binder, and the electrical conductor on the positive electrode current collector, to thereby form the positive electrode active material layer on the positive electrode current collector, and rolling the positive electrode active material layer.

Examples of the positive electrode active material include a lithium transition metal oxide, and specific examples thereof include a lithium cobalt composite oxide, a lithium manganese composite oxide, a lithium nickel composite oxide, a lithium nickel manganese composite oxide, and a lithium nickel cobalt composite oxide. These may be used singly or in combinations of two or more thereof.

A positive electrode active material containing a lithium nickel composite oxide as a main component can make the capacity of a non-aqueous electrolyte secondary battery large, but is likely to produce side reaction products attributable to nickel, and therefore the decrease in the capacity recovery rate of a non-aqueous electrolyte secondary battery after high-temperature storage is likely to be brought about. The main component refers to a component the content of which is the largest among the materials constituting a positive electrode active material.

However, the non-aqueous electrolyte containing the above fluorine-containing cyclic carbonate, the above imide lithium salt having sulfonyl groups, and the above cyclic carboxylic anhydride, as compared to a non-aqueous electrolyte not containing any one of the above three types of substances, can suppress the production of the side reaction products attributable to nickel. That is, both the large capacity of a non-aqueous electrolyte secondary battery and the suppression of the decrease in the capacity recovery rate of a non-aqueous electrolyte secondary battery after high-temperature storage can be achieved by a combination of the non-aqueous electrolyte and the positive electrode active material containing a lithium nickel composite oxide as a main component according to the present embodiment.

For example, the content of the lithium nickel composite oxide in the positive electrode active material is preferably 50 mass % or more, and more preferably 80 mass % or less. If the content of the lithium nickel composite oxide in the positive electrode active material is less than 50 mass %, as compared to a case where the content meets the above range, the capacity of the non-aqueous electrolyte secondary battery may decrease. The lithium nickel composite oxide alone can be used as the positive electrode active material.

The lithium nickel composite oxide is not particularly limited as long as it is an oxide containing lithium and nickel; however, for example, in light of enabling the large capacity of the non-aqueous electrolyte secondary battery, a lithium nickel composite oxide having a ratio of nickel to the total number of moles of metal elements excluding lithium of 20 mol % or more is preferable, and more preferably a lithium nickel composite oxide represented by a general formula LiₓNi_{y}M_{(1-y)}O₂ {0.1≤x≤1.2; 0.2≤y≤1; and M represents at least one metal element}. Examples of M include Co, Mn, Mg, Zr, Al, Cr, V, Ce, Ti, Fe, K, Ga, and In. Among these, M preferably includes at least one of cobalt (Co), manganese (Mn), and aluminum (Al), and more preferably includes Co and Al from the viewpoints such as the large capacity of the non-aqueous electrolyte secondary battery.

Examples of the electrical conductor include carbon powders such as carbon black, acetylene black, Ketjenblack, and graphite. These may be used singly or in combinations of two or more thereof.

Examples of the binder include fluoropolymers, and rubber polymers. Examples of the fluoropolymers include polytetrafluoroethylene (PTFE), poly (vinylidene fluoride) (PVdF), and modified products thereof and examples of the rubber polymers include ethylene-propylene-isoprene copolymers and ethylene-propylene-butadiene copolymers. These may be used singly or in combinations of two or more thereof.

[Negative Electrode]

The negative electrode includes, for example, a negative electrode current collector such as metal foil and a negative electrode active material layer formed on the negative electrode current collector. Foil of a metal, such as copper, that is stable in the electric potential range of the negative electrode, a film with such a metal disposed as an outer layer, and the like can be used for the negative electrode current collector. The negative electrode active material layer contains, for example, a negative electrode active material, a binder, a thickener, and the like.

The negative electrode is obtained by, for example, applying/drying a negative electrode mixture slurry containing the negative electrode active material, the thickener, and the binder on the negative electrode current collector, to thereby form the negative electrode active material layer on the negative electrode current collector, and rolling the negative electrode active material layer.

The negative electrode active material is not particularly limited as long as it is a material that can intercalate and deintercalate lithium ions, and examples thereof include metal lithium; lithium alloys such as lithium-aluminum alloy, lithium-lead alloy, lithium-silicon alloy, and lithium-tin alloy; carbon materials such as graphite, cokes, and organic substance-fired products; and metal oxides such as SnO₂, SnO, and TiO₂. These may be used singly or in combinations of two or more thereof.

As the binder, for example, a fluorine polymer, a rubber polymer, or the like can be used in the same manner as in the case of the positive electrode; however, a styrene-butadiene copolymer (SBR) or a modified product thereof may also be used.

Examples of the thickener include carboxymethyl cellulose (CMC) and poly(ethylene oxide) (PEO). These may be used singly or in combinations of two or more thereof.

[Separator]

For example, an ion-permeable and insulating porous sheet or the like is used as the separator. Specific examples of the porous sheet include a microporous thin film, woven fabric, and nonwoven fabric. Suitable examples of the material for the separator include olefin resins such as polyethylene and polypropylene, and cellulose. The separator may be a laminate including a cellulose fiber layer and a layer of fibers of a thermoplastic resin such as an olefin resin. The separator may be a multi-layered separator including a polyethylene layer and a polypropylene layer, and a separator a surface of which is coated with a material such as an aramid resin or a ceramic may also be used as the separator.

### EXAMPLES

Hereinafter, the present disclosure will be further described by way of Examples, but is not limited to the following Examples.

### Example 1

[Production of Positive Electrode]

A lithium complex oxide represented by a general formula LiNi_{0.8}Co_{0.15}Al_{0.15}O₂ was used as a positive electrode active material. The positive electrode active material, acetylene black as an electrical conductor, and poly (vinylidene fluoride) as a binder were mixed so that the contents thereof were 100 mass %, 1 mass %, and 0.9 mass % respectively, and N-methyl-2-pyrrolidone (NMP) was added thereto to prepare a positive electrode mixture slurry. Subsequently, the positive electrode mixture slurry was applied on each side of an aluminum positive electrode current collector having a thickness of 15 μm by a doctor blade method, and the resulting applying film was rolled, to thereby form a positive electrode active material layer having a thickness of 70 μm on each side of the positive electrode current collector. This was used as a positive electrode.

[Production of Negative Electrode]

Graphite as a negative electrode active material, carboxymethyl cellulose (CMC) as a thickener, and a styrene-butadiene copolymer (SBR) as a binder were mixed so that the contents thereof were 100 mass %, 1 mass %, and 1 mass % respectively, and water was added thereto to prepare a negative electrode mixture slurry. Subsequently, the negative electrode mixture slurry was applied on each side of a copper negative electrode current collector having a thickness of 10 μm by a doctor blade method, and the resulting applying film was rolled, to thereby form a negative electrode active material layer having a thickness of 80 μm on each side of the negative electrode current collector. This was used as a negative electrode.

[Preparation of Non-Aqueous Electrolyte]

In a mixed solvent obtained by mixing monofluoroethylene carbonate (FEC), ethyl methyl carbonate (EMC), and dimethyl carbonate (DMC) in a volume ratio of 15:45:40, LiPF₆ was dissolved at a concentration of 1.3 mol/L, and further, diglycolic anhydride (DGA) and lithium bis(fluorosulfonyl)imide (LiFSI) were dissolved so that the contents thereof were 0.5 mass % and 0.5 mass % respectively, to thereby prepare a non-aqueous electrolyte.

[Production of Non-Aqueous Electrolyte Secondary Battery]

The above positive electrode and negative electrode were each cut into a predetermined size, and the resulting electrodes were each attached to an electrode tab and wound through a separator, to thereby produce a wound type electrode assembly. Subsequently, the electrode assembly was housed in an aluminum laminate film, and the above electrolyte solution was injected thereinto to be sealed. This was used as a non-aqueous electrolyte secondary battery of Example.

### Comparative Example 1

A non-aqueous electrolyte was prepared in the same manner as in Example 1, except that diglycolic anhydride and lithium bis(fluorosulfonyl)imide were not added in the preparation of a non-aqueous electrolyte. Subsequently, a non-aqueous electrolyte secondary battery was produced using the non-aqueous electrolyte in the same manner as in Example 1.

### Comparative Example 2

A non-aqueous electrolyte was prepared in the same manner as in Example 1, except that lithium bis(fluorosulfonyl)imide was not added in the preparation of a non-aqueous electrolyte. A non-aqueous electrolyte secondary battery was produced using the non-aqueous electrolyte in the same manner as in Example 1.

### Comparative Example 3

A non-aqueous electrolyte was prepared in the same manner as in Example 1, except that diglycolic anhydride was not added in the preparation of a non-aqueous electrolyte. A non-aqueous electrolyte secondary battery was produced using the non-aqueous electrolyte in the same manner as in Example 1.

[Measurement of Capacity Recovery Rate after High-Temperature Storage]

Measurement of the capacity recovery rate after high-temperature storage was performed for the non-aqueous electrolyte secondary batteries of Example and Comparative Examples under the following condition. At an environmental temperature of 25° C., a charging was carried out at a constant current of 0.5 lt to a voltage of 4.1 V, and thereafter a constant voltage charging at 4.1 V was carried out to a current of 0.05 lt, to complete charge (the charge is referred to as charge A). After a quiescent period of 10 minutes, a constant current discharging was carried out at a constant current of 0.5 lt to 3.0 V (the discharge is referred to as discharge A), and the discharge capacity on that occasion was defined as the capacity before storage. After a quiescent period of 10 minutes, only the above charge A was performed, and thereafter the non-aqueous electrolyte batteries were stored at an environmental temperature of 45° C. for 15 days. The temperature was decreased to room temperature after the storage, and thereafter only the above discharge A was performed. The above charge A was performed after a quiescent period of 10 minutes, and thereafter the above discharge A was performed after a quiescent period of 10 minutes, and the discharge capacity on that occasion was defined as a recovered capacity. The capacity recovery rate after high-temperature storage was determined from the following formula.

Capacity recovery rate after high-temperature storage (%)=recovered capacity/capacity before storage×100

[Measurement of Amount of Gas Produced after High-Temperature Storage]

The volume A (mL) of each of the non-aqueous electrolyte secondary batteries of Example and Comparative Examples was measured by an Archimedes method. The above charge A was performed for each non-aqueous electrolyte secondary battery, and after the non-aqueous electrolyte secondary batteries were stored at an environmental temperature of 45° C. for 15 days, the volume B (mL) of each non-aqueous electrolyte secondary battery was measured by the Archimedes method. The amount of a gas produced after high-temperature storage was calculated by subtracting the volume A (mL) from the volume B (mL). When the amount of a gas produced in Comparative Example 1 is defined as a standard (100%), the relative ratio of the amount of a gas produced in the non-aqueous electrolyte secondary battery of each of Example and the other Comparative Examples is defined as the gas production amount ratio. The Archimedes method refers to a method in which a measuring object (non-aqueous electrolyte secondary battery) is immersed in a medium (e.g. distilled water or alcohol) to measure the buoyancy to which the measuring object is subjected, to thereby measure the volume of the measuring object.

[Charge/Discharge Cycle Test]

At an environmental temperature of 25° C., a constant current charging was carried out at a constant current of 0.5 lt to a voltage of 4.1 V, and thereafter a constant current discharging was carried out at a constant current of 0.5 lt to a voltage of 3.0 lt for each of the non-aqueous electrolyte secondary batteries of Example and Comparative Examples. 75 cycles of the charging/discharging were performed. The capacity retention rate was determined from the following formula. It is indicated that the higher this value is, the more the deterioration in the charge/discharge cycle characteristics is suppressed.

Capacity retention rate=(discharge capacity at 75th cycle/discharge capacity at first cycle)×100

Table 1 shows the content of monofluoroethylene carbonate (FEC), the content of diglycolic anhydride (DGA), and the content of lithium bis(fluorosulfonyl)imide (LiFSI) in the non-aqueous electrolytes used in Example and Comparative Examples 1 to 3, and the results of the capacity recovery rate after high-temperature storage, the gas production amount ratio, and the capacity retention rate at the time of performing 75 cycles of charging/discharging for the non-aqueous electrolyte secondary batteries of Example and Comparative Examples.

TABLE 1

| | | | | High-temperature storage | | Capacity retention rate at the |
|---|---|---|---|---|---|---|
| | Non-aqueous electrolytes | | | Capacity recovery | Gas production | time of performing 75 cycles |
| | FEC content | DGA content | LiFSI content | rate | amount ratio | of charging/discharging |
| Example | 15 vol % | 0.5 mass % | 0.5 mass % | 98.6% | 91.6% | 98.6% |
| Comparative | 15 vol % | — | — | 95.8% | 100.0% | 98.5% |
| Example 1 | | | | | | |
| Comparative | 15 vol % | 0.5 mass % | — | 97.7% | 112.3% | 98.7% |
| Example 2 | | | | | | |
| Comparative | 15 vol % | — | 0.5 mass % | 96.7% | 97.4% | 97.9% |
| Example 3 | | | | | | |

FEC: monofluoroethylene carbonate
DGA: diglycolic anhydride
LiFSI: lithium bis(fluorosulfonyl)imide

The non-aqueous electrolyte secondary battery of Example, using the non-aqueous electrolyte containing a fluorine-containing cyclic carbonate; the cyclic carboxylic anhydride represented by the above formula (1); and the imide lithium salt having sulfonyl groups and represented by the above formula (2), as compared to the non-aqueous electrolyte secondary batteries of Comparative Examples 1 to 3 each using a non-aqueous electrolyte not containing at least any one of the cyclic carboxylic anhydride represented by the above formula (1) and the imide lithium salt having sulfonyl groups and represented by the above formula (2), exhibits a high capacity recovery rate after high-temperature storage, a low gas production amount ratio, and at least an equal capacity retention rate at the time of performing 75 cycles of charging/discharging.

1. A non-aqueous electrolyte secondary battery comprising:
a positive electrode;
a negative electrode; and
a non-aqueous electrolyte, wherein
the non-aqueous electrolyte contains:
a non-aqueous solvent containing a fluorine-containing cyclic carbonate;
a cyclic carboxylic anhydride represented by the following formula (1): 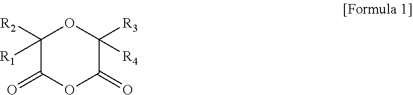
wherein R₁ to R₄ each independently represent H, an alkyl group, an alkene group, or an aryl group; and
an imide lithium salt having sulfonyl groups and represented by the following formula (2): 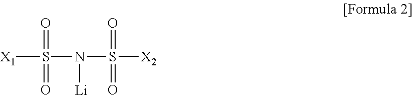
wherein X₁ to X₂ each independently represent a fluorine group or a fluoroalkyl group.

2. The non-aqueous electrolyte secondary battery according to claim 1, wherein the cyclic carboxylic anhydride contains at least one of diglycolic anhydride, methyldiglycolic anhydride, dimethyldiglycolic anhydride, ethyldiglycolic anhydride, vinyldiglycolic anhydride, allyldiglycolic anhydride, and divinyldiglycolic anhydride.

3. The non-aqueous electrolyte secondary battery according to claim 1, wherein the imide lithium salt having sulfonyl groups contains at least one of lithium bis(fluorosulfonyl)imide, lithium bis(trifluoromethanesulfonyl)imide, lithium bis(pentafluoroethanesulfonyl)imide, and lithium bis(nonafluorobutanesulfonyl)imide.

4. The non-aqueous electrolyte secondary battery according to claim 1, wherein the content of the fluorine-containing cyclic carbonate in the non-aqueous solvent is 5 vol % or more and 50 vol % or less.

5. The non-aqueous electrolyte secondary battery according to claim 1, wherein the content of the cyclic carboxylic anhydride in the non-aqueous electrolyte is 0.1 mass % or more and 1.5 mass % or less, and the content of the imide lithium salt having sulfonyl groups in the non-aqueous electrolyte is 0.1 mass % or more and 1.5 mass % or less.
